(19) United States
(12) Patent Application Publication
(10) Pub. No.: US 2016/0143850 A1
(43) Pub. Date: May 26, 2016

(54) Oral Pharmaceutical Compositions Comprising Imatinib Mesylate

(71) Applicant: SHILPA MEDICARE LIMITED, Raichur (IN)

(72) Inventors: PRADEEP SHIVAKUMAR, VIZIANAGARAM (IN); KRISHNAMURTHY TOPPALADODDI, VIZIANAGARAM (IN); SANJAY UMACHIGI, VIZIANAGARAM (IN); BADRINATH ALAMPALLI, RAICHUR (IN); AKSHAYKANT CHATURVEDI, RAICHUR (IN)

(73) Assignee: SHILPA MEDICARE LIMITED, RAICHUR, OT (IN)

(21) Appl. No.: 14/901,079

(22) PCT Filed: Jun. 25, 2014
(86) PCT No.: PCT/IB2014/062583

§ 371 Date: Dec. 28, 2015

(30) Foreign Application Priority Data
Jul. 9, 2013 (IN) 3059/CHE/2013

(51) Int. Cl. A61K 9/16; A61K 9/28; A61K 9/20; A61K 31/506

(52) U.S. Cl. CPC A61K 9/1635; A61K 31/506; A61K 9/1617; A61K 9/1682; A61K 9/28; A61K 9/2095; A61K 9/2027; A61K 9/2013

(57) ABSTRACT

The invention relates to a granulate composition comprising 90-99.95% w/w of Imatinib mesylate, 0.05-0.2% w/w of binder and 0-8% w/w of disintegrant, wherein the granulate composition is prepared using binder in isopropyl alcohol solvent.

The invention also relates to pharmaceutical compositions comprising Imatinib mesylate, with high polymorphic stability and processes for preparation thereof.

Said granulate composition comprising 90-99.95% w/w of Imatinib mesylate and their pharmaceutical compositions derived may be useful in the treatment of cancer.

The following specification particularly describes the invention and the manner in which it is to be performed.

### FIELD OF THE INVENTION

The invention relates to a granulate composition comprising 90-99.95% w/w of imatinib mesylate, 0.05-0.2% w/w of binder and 0-8% w/w of disintegrant.

The invention also relates to pharmaceutical compositions comprising a granulate composition of Imatinib mesylate and processes for preparation thereof.

The pharmaceutical compositions derived from a granulate composition comprising 90-99.95% w/w of Imatinib mesylate are useful in the treatment of cancer.

### BACKGROUND OF THE INVENTION

Imatinib mesylate (I) is chemically known as 4-[(4-Methyl-1-piperazinyl)methyl]-N-[4-methyl-3-[[4-(3-pyridinyl)-2-pyrimidinyl]amino]-phenyl]benzamide methanesulfonate.

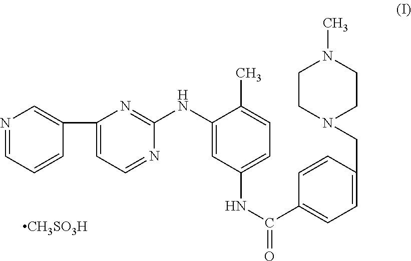

Imatinib mesylate is a tyrosine-kinase inhibitor used in the treatment of multiple cancers and is sold under the trade name GLEEVEC®/GLIVEC®. Imatinib is used in chronic myelogenous leukemia (CML), gastrointestinal stromal tumors (GISTs) and a number of other malignancies. The U.S. Food and Drug Administration (FDA) have approved Imatinib as first-line treatment for Philadelphia chromosome positive (Ph+) Chronic Myelogenous Leukemia (CML), both in adults and children. The drug is approved in multiple Ph+ cases CML, including after stem cell transplant, in blast crisis, and newly diagnosed. The FDA first granted approval for advanced GIST patients in 2002. Later in 2012, Imatinib was approved also for use after the surgical removal of KIT-positive tumors to help prevent recurrence. The drug is also approved in unresectable KIT-positive GISTs.

USFDA has approved imatinib for use in adult patients with relapsed or refractory Ph-positive Acute lymphoblastic leukemia (ALL), myelodysplastic/myeloproliferative diseases associated with platelet-derived growth factor receptor gene re-arrangements, aggressive systemic mastocytosis (ASM) without or an unknown D816V c-KIT mutation, hypereosinophilic syndrome (HES) and/or chronic eosinophilic leukemia (CEL) who have the FIP1L1-PDGFRα fusion kinase (CHIC2 allele deletion) or FIP1L1-PDGFRα fusion kinase negative or unknown, unresectable, recurrent and/or metastatic dermatofibrosarcoma protuberans. Recently on 25 Jan. 2013, Gleevec has been approved for use in children with Ph+ ALL.

Imatinib and its salts have been disclosed in EP0564409B1 (U.S. Pat. No. 5,521,184). Specifically, imatinib mesylate and its crystalline polymorphic Forms α and β are disclosed in WO99/03854A1.

Furthermore, EP564409B1 and WO99/03854A1 disclose generically many possible formulations, which are prepared in a manner known per se, for example by means of conventional mixing, granulating, dissolving or lyophilizing processes, and comprise approximately from 1% to 100%, especially from approximately 1% to approximately 20%, active ingredient. In the examples, direct compression, gelatinizing and mixing techniques are disclosed.

One of the anonymously filed and published literature in IP.com (on Feb. 19, 2008 of IP.com number: IPCOM000167554D) discloses stable tablet formulation containing more than 80% of Imatinib mesylate.

Wet granulation process for making imatinib pharmaceutical compositions has been disclosed by Luftensteiner et al in patent application EP1501485B1 (WO03090720A1). Imatinib mesylate (Form α and β disclosed) and binder are mixed together with water and the mixture is processed for granulation, e.g., using a high-shear granulator to form wet granulates containing 30% to 80% imatinib mesylate and binder.

Malhotra et al in WO2012080703A1 disclose a solid oral pharmaceutical composition and its granulate comprises greater than 100 mg of imatinib, and one or more pharmaceutically acceptable excipients.

Adhibatla et al in WO2012176014A1 disclose an oral pharmaceutical composition comprising greater than 80% Imatinib by weight based on the total weight of the composition.

Konatham et al in WO2013008253A2 disclose a stable pharmaceutical formulation comprising imatinib or pharmaceutically acceptable salt thereof comprising one or more pharmaceutically acceptable excipients.

Van Den et al in WO2012/019633A1 disclose a wet granulation process for making imatinib pharmaceutical compositions which comprise a process without the use of additional excipients, such as binders, except for the granulation liquid.

Bilgic et al in WO2012/087255A2 covers a pharmaceutical formulation comprising imatinib characterized in that said formulation comprises a pharmaceutically acceptable disintegrant in the range of 0.1% to 5% by weight and at least one other excipient.

Bilgic et al in WO2012/087256A2 covers pharmaceutical formulations comprising imatinib characterized in that said formulations comprise a pharmaceutically acceptable lubricant, a glidant and at least one other excipient; and the ratio of the lubricant and the glidant comprised in the formulations to each other is minimum 5 by weight.

Zimmermann et al in EP564409 B1 initially disclosed the preparation of Imatinib in free form (not as a salt). Further, Zimmermann et al in U.S. Pat. No. 6,894,051B1 described α and β crystal forms of Imatinib Mesylate.

Zimmermann et al in U.S. '051 disclosed that the α-crystal form of 4-(4-methylpiperazin-1-ylmethyl)-N-[4-methyl-3-(4-pyridin-3-yl)pyrimidin-2-ylamino)phenyl]benzamide methane sulfonate i.e. Imatinib mesylate is characterized by needle-shaped crystals and is hygroscopic. It has also mentioned that in this form, the crystals are not particularly well-suited to pharmaceutical formulation as solid dosage forms, because their physical properties, for example their flow characteristics, are unfavorable and unsuitable.

It was further emphasized that under certain conditions, however, it is possible to obtain 4-(4-methylpiperazin-1-ylmethyl)-N-[4-methyl3-(4-pyridin-3-yl)pyrimidin-2-yl amino)phenyl]benzamide methane sulfonate in a crystal form which is not needle-shaped, this crystal form being described as β-crystal form. The β-crystal form of Imatinib mesylate is detailed as having the advantage of its flow properties being substantially more favorable than those of the α-crystal form. This crystal form has the further advantage of being thermodynamically more stable at temperatures below 140° C. Also applicant of U.S. '051 describes that the β-crystal form is less hygroscopic than the α-crystal form and thus also stores better and is easier to process.

Besides α and β crystal forms various other polymorphic forms of Imatinib mesylate and the process for preparation thereof have been described in patent publications WO2004/106326, WO2005/077933, WO2005/095379, WO2006/054314, WO2006/0223816, WO2006/048890, WO2007/023182, WO2007/059963 and WO2011/108953.

Gerber et al in U.S. Pat. No. 8,414,918 purportedly discloses a pharmaceutical composition in the form of a coated tablet comprising polymorphic form X of imatinib mesylate, wherein less than 10% of the polymorphic form of imatinib mesylate is converted to form α or form β after storage at 40° C. at 75% relative humidity for 1 month, and wherein the coated tablet is prepared by coating a tablet using a C₁₋₄ alcohol solvent with less than 20% w/v water. In Gerber et al, the uncoated pharmaceutical composition, such as a tablet, is prepared by dry granulation or direct compression.

The inventors of 2548/CHE/2013 application have developed a process which provides a stable polymorphic crystalline form of Imatinib mesylate, designated as Form-SA, which is non-hygroscopic, non-needle shaped and thus has easy handling properties. The process of this invention provides the crystalline Form-SA of Imatinib mesylate in a substantially pure form, which is without any detectable impurities/contamination of any other previously known crystalline forms of Imatinib mesylate. The invention relates to crystalline Form-SA obtained by the process of the present invention, the said Form-SA being substantially pure, stable, non-needle shaped and characterized by X-ray powder diffraction pattern comprising of at least five 2θ° peaks selected from 10.67, 12.90, 15.34, 19.49, 19.80, 26.06, 26.32 and 28.89±0.05 2θ°.

Several granulation processes have been reported in the prior art however, due to unsuitable and non-up scalable tableting properties of imatinib mesylate and its reported formulations itself, there remains a need for the composition/formulation, which is not only up-scalable on industrial scale but also provides a stable composition useful in the treatment of cancer. Hence, an improvement in this field, in particular by finding a simple and easily scalable process of improving granulating and tabletting properties of imatinib mesylate and retaining high polymorphic stability of imatinib mesylate in pharmaceutical compositions is still desirable.

### SUMMARY OF INVENTION

Provided herein are simple and easily scalable oral pharmaceutical compositions of imatinib mesylate with improved granulating, tabletting or encapsulation properties, and the process of preparing such composition. The present invention provides a tablet containing imatinib, preferably imatinib mesylate, which provides high polymorphic stability of imatinib mesylate within the compositions.

Aspects of the present invention relates to a granulate composition comprising 90-99.95% w/w of imatinib mesylate, 0.05-0.2% w/w of binder and 0-8% w/w of disintegrant.

Aspects of the present invention relates to a granulate composition comprising 95-99.95% w/w of imatinib mesylate, 0.05-0.2% w/w of binder and 0-5% w/w of disintegrant.

Aspects of the present invention relates to a granulate composition comprising 95-99.95% w/w of imatinib mesylate, 0.05-0.2% w/w of binder and 0-5% w/w of disintegrant, wherein the granulate composition is prepared using binder in isopropyl alcohol solvent.

Aspects of the present invention relates to a granulate composition comprising 95-99.95% w/w of imatinib mesylate, 0.05-0.1% w/w of binder and 0% w/w of disintegrant.

Aspects of the present invention relates to a granulate composition comprising imatinib mesylate crystalline Form-SA (I), which is stable, non-needle shaped and characterized by X-ray powder diffraction pattern comprising of at least five 2θ° peaks selected from 10.67, 12.90, 15.34, 19.49, 19.80, 26.06, 26.32 and 28.89+0.1 2θ°.

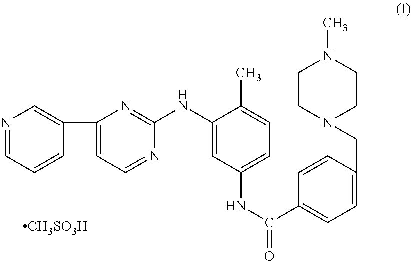

Aspects of the present invention relates to a pharmaceutical composition comprising crystalline non-needle shaped Form-SA of Imatinib mesylate and at least one or more pharmaceutically acceptable excipients. Such composition is substantially free of any other previously known crystalline forms of Imatinib mesylate.

Aspects of the present invention relates to a granulate containing 90-99.95% w/w of imatinib mesylate, 0.05-0.2% w/w of polyvinyl pyrrolidone and 0-8% w/w of crospovidone, wherein the granulate is prepared by process comprising:
a) providing a solution of polyvinyl pyrrolidone in isopropyl alcohol;
b) wetting imatinib mesylate and crospovidone mixture with step a) solution, and
c) granulating the mixture in a granulator, followed by drying, and optionally sieving and/or milling.

Aspects of the present invention relates to a process for making the pharmaceutical composition for oral administration comprising a step of admixing the imatinib mesylate granulate with pharmaceutically acceptable excipients, optionally, comprising a step of compressing the composition into tablets, and optionally followed by a film-coating.

Aspects of the present invention further relates to a process for preparing a pharmaceutical composition, preferably a tablet, containing imatinib mesylate, wherein the pharmaceutical composition provides high polymorphic stability comprising: coating a pharmaceutical composition, preferably a tablet, comprising crystalline imatinib, with a coating solution, preferably a tablet coating solution, containing an C₁₋₄ alcohol solvent with an amount of less than about 10% v/v of water, preferably less than 5% v/v.

Aspects of the present invention also relates to a solid oral pharmaceutical composition of imatinib mesylate comprising: Imatinib mesylate: 96-99% w/w, Binder: 0.05-0.2% w/w, Disintegrant: 0-5% w/w, Lubricant: 0.2-0.5% w/w, Film coating: 1-3% w/w; wherein the % w/w is relative to the total weight of pharmaceutical composition.

Aspects of the present invention relates to a granulate and/or the pharmaceutical composition of imatinib mesylate for use in medicine, such as the treatment of various types of cancer diseases.

Further, particular aspects of the invention are detailed in the description part of the specification, wherever appropriate.

### BRIEF DESCRIPTION OF THE DRAWINGS

FIG. 1 is X-ray powder diffraction (“XRPD”) pattern of crystalline Form-SA of Imatinib mesylate.

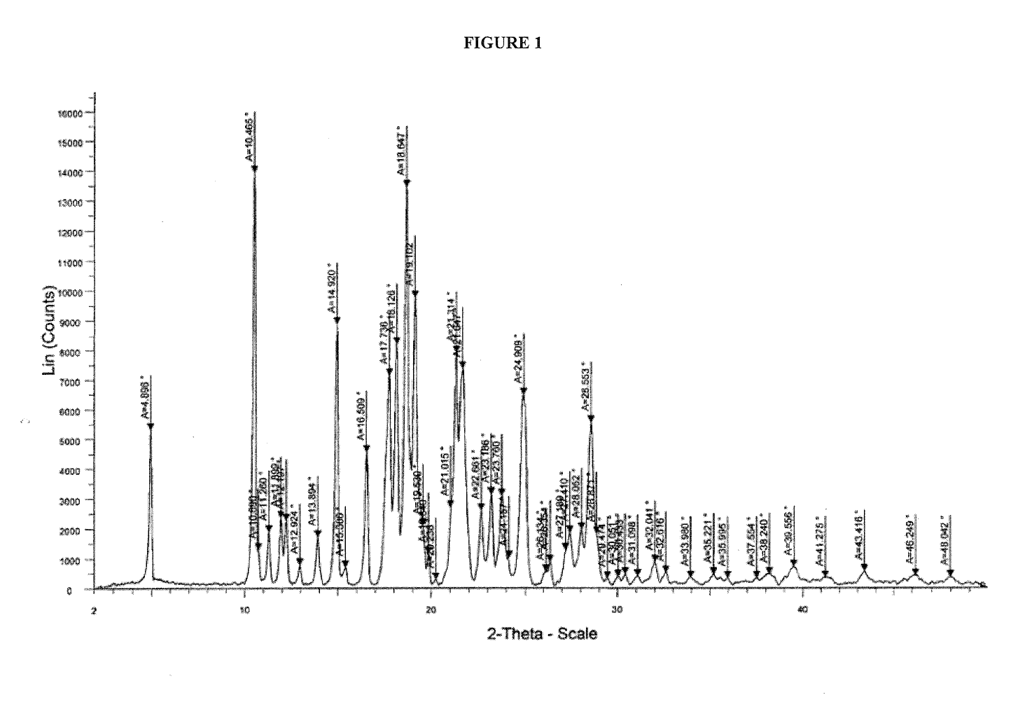

FIG. 2 is an X-ray powder diffraction (“XRPD”) pattern of example 1B formulation.

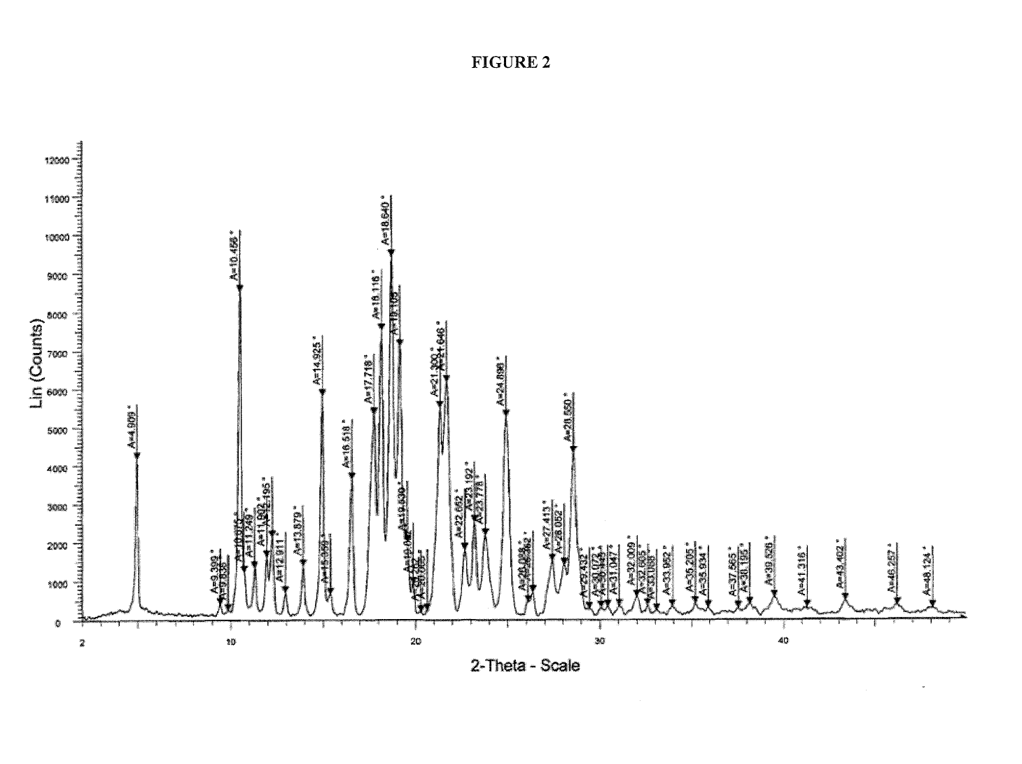

FIG. 3 is an X-ray powder diffraction (“XRPD”) pattern of example 7 initial formulation.

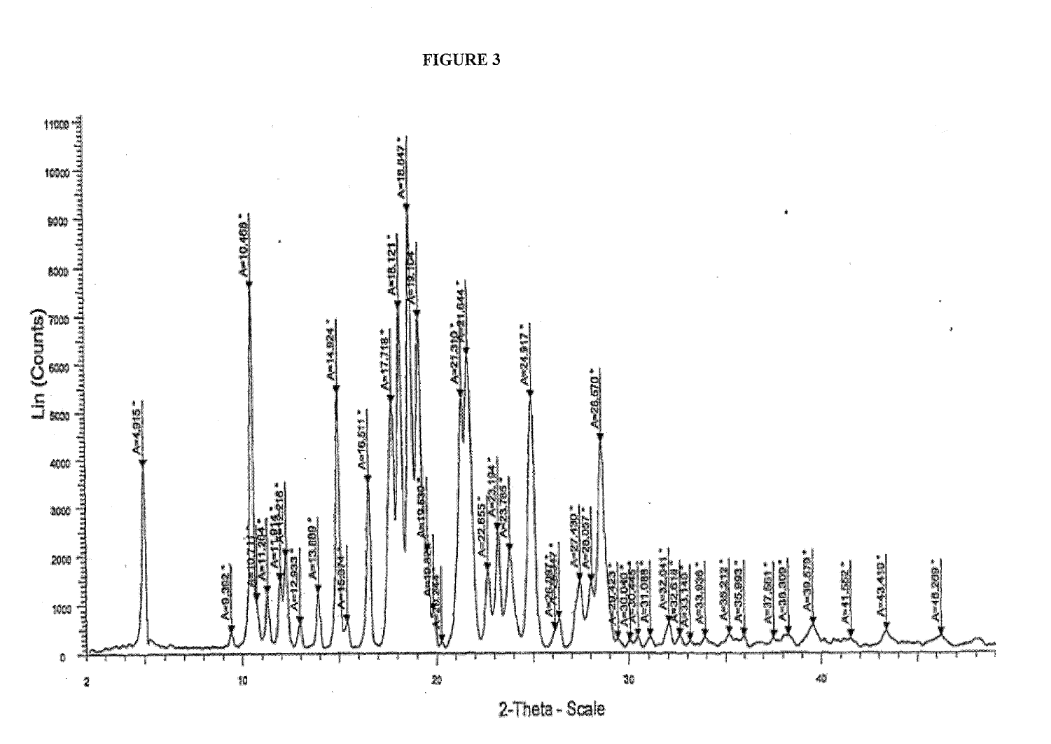

FIG. 4 is an X-ray powder diffraction (“XRPD”) pattern of example 7 formulation OPD 2M.

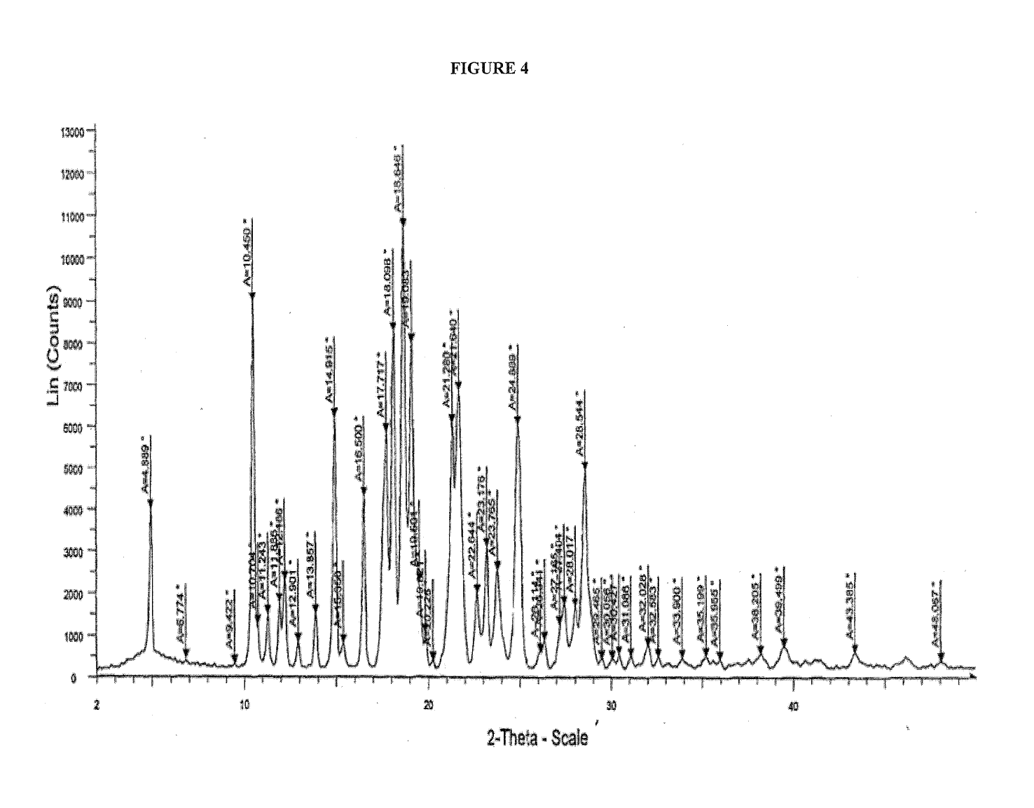

Table below provides comparative XRD profiles of Imatinib mesylate Form-SA & Example 1B Imatinib mesylate Film coated tablets (FCT)—Prepared by IPA granulation and Aqueous Film Coating.

| Form-SA of Imatinib mesylate | Example 1B Film Coated tablets |
|---|---|
| 4.896 | 4.909 |
| 10.465 | 9.399 |
| 10.69 | 9.838 |
| 11.26 | 10.46 |
| 11.899 | 10.68 |
| 12.197 | 11.25 |
| 12.924 | 11.9 |
| 13.894 | 12.2 |
| 14.92 | 12.91 |
| 15.386 | 13.88 |
| 16.509 | 14.93 |
| 17.736 | 15.36 |
| 18.126 | 16.52 |
| 18.647 | 17.72 |
| 19.102 | 18.12 |
| 19.53 | 18.64 |
| 19.84 | 19.53 |
| 20.238 | 19.11 |
| 21.015 | 19.85 |
| 21.314 | 20.26 |
| 21.647 | 20.61 |
| 22.661 | 21.3 |
| 23.186 | 21.65 |
| 23.76 | 22.65 |
| 24.157 | 23.19 |
| 24.909 | 24.9 |
| 26.134 | 26.09 |
| 26.354 | 23.78 |
| 27.189 | 26.35 |
| 27.41 | 27.41 |
| 28.052 | 28.05 |
| 28.553 | 28.55 |
| 28.871 | 29.43 |
| 29.474 | 30.07 |
| 30.051 | 30.45 |
| 30.433 | 31.05 |
| 31.098 | 32.01 |
| 32.041 | 32.61 |
| 32.616 | 33.09 |
| 33.98 | 33.95 |
| 35.221 | 35.21 |
| 35.995 | 35.93 |
| 37.554 | 37.57 |
| 38.24 | 38.20 |
| 39.556 | 39.53 |
| 41.275 | 41.32 |
| 43.416 | 43.4 |
| 46.249 | 46.26 |
| 48.042 | 48.12 |

The XRD spectrum of processed placebo of Example 1B show only few significant peaks at 3.537, 5.403, 9.524 & 25.309, which do not relates to any peaks to the corresponding XRD patterns of Imatinib mesylate Form-SA and Imatinib mesylate FCT.

Table below provides comparative XRD profiles of Imatinib mesylate Form-SA & Example 7 Imatinib mesylate Film coated tablets (FCT)—Prepared by IPA granulation and mixture of IPA-water Film Coating.

| | Example 7 | Example 7 |
|---|---|---|
| Imatinib mesylate | Film Coated | Film Coated |
| Form-SA | tablets (Initial) | tablets (OPD 2M) |
| 4.924 | 4.915 | 4.889 |
| 10.485 | 9.392 | 6.774 |
| 10.685 | 10.468 | 9.422 |
| 11.278 | 10.711 | 10.45 |
| 11.905 | 11.264 | 10.704 |
| 12.217 | 11.914 | 11.243 |
| 12.939 | 12.216 | 11.886 |
| 13.891 | 12.933 | 12.186 |
| 14.927 | 13.889 | 12.901 |
| 15.383 | 14.924 | 13.857 |
| 16.513 | 15.374 | 14.915 |
| 17.749 | 16.511 | 15.35 |
| 18.122 | 17.718 | 16.5 |
| 18.653 | 18.121 | 17.717 |
| 19.102 | 18.647 | 18.098 |
| 19.53 | 19.104 | 18.646 |
| 19.846 | 19.53 | 19.083 |
| 20.244 | 19.827 | 19.501 |
| 21.308 | 20.244 | 19.821 |
| 21.633 | 21.31 | 20.225 |
| 22.661 | 21.644 | 21.28 |
| 23.193 | 22.655 | 21.64 |
| 23.766 | 23.194 | 22.644 |
| 24.911 | 23.785 | 23.176 |
| 26.108 | 24.917 | 23.765 |
| 26.367 | 26.097 | 24.889 |
| 27.189 | 26.347 | 26.114 |
| 27.433 | 27.43 | 26.341 |
| 28.053 | 28.057 | 27.165 |
| 28.541 | 28.57 | 27.404 |
| 28.907 | 29.423 | 28.017 |
| 29.508 | 30.04 | 28.544 |
| 30.037 | 30.445 | 29.465 |
| 30.462 | 31.088 | 30.056 |
| 31.112 | 32.041 | 30.427 |
| 31.56 | 32.618 | 31.086 |
| 32.041 | 33.14 | 32.028 |
| 32.614 | 33.936 | 32.583 |
| 33.091 | 35.212 | 33.90 |
| 33.952 | 35.993 | 35.199 |
| 35.192 | 37.561 | 35.955 |
| 35.98 | 38.309 | 38.205 |
| 37.537 | 39.579 | 39.499 |
| 38.246 | 41.552 | 43.385 |
| 39.585 | 43.41 | 48.067 |
| 41.491 | 46.269 | — |
| 43.405 | — | — |
| 45.654 | — | — |
| 46.18 | — | — |
| 48.026 | — | — |
| 48.734 | — | — |

The XRD spectrum of processed placebo of Example 8 show only few significant peaks at 5.208, 9.427, 20.794, 21.225, 21.812, 23.418, 25.258, 29.615; which do not relates to any peaks to the corresponding XRD patterns of Imatinib mesylate Form-SA and Imatinib mesylate FCT.

### Abbreviations

| | |
|---|---|
| API | Active Pharmaceutical Ingredient |
| DSC | Differential Scanning Calorimetry |
| HPLC | High-Performance Liquid Chromatography |
| IPA | Iso-Propyl Alcohol or isopropanol |
| RPM | Rotations Per Minute |
| XRPD | X-Ray Powder Diffraction |
| LOD | Loss On Drying |
| OPD | Open Petri Dish |

### DETAILED DESCRIPTION

In one of the embodiment according to present invention, it provides a granulate composition comprising 90-99.95% w/w of imatinib mesylate, 0.05-0.2% w/w of binder and 0-8% w/w of disintegrant.

In one of the embodiment according to present invention, it provides a granulate composition comprising 90-99.95% w/w of imatinib mesylate, 0.05-0.2% w/w of binder and 0-8% w/w of disintegrant, wherein the granulate composition is prepared using binder in isopropyl alcohol solvent.

In one of the preferred embodiments according to present invention, it provides a granulate composition comprising 95-99.95% w/w of imatinib mesylate, 0.05-0.2% w/w of binder and 0-5% w/w of disintegrant.

In one of the preferred embodiments according to present invention, it provides a granulate composition comprising 95-99.95% w/w of imatinib mesylate, 0.05-0.2% w/w of binder and 0-5% w/w of disintegrant, wherein the granulate composition is prepared using binder in isopropyl alcohol solvent.

In one of the preferred embodiments according to present invention, it provides a granulate composition comprising 95-99.95% w/w of imatinib mesylate, 0.05-0.1% w/w of binder and 0% w/w of disintegrant.

In one of the preferred embodiments according to present invention, it provides a granulate composition comprising 95-99.95% w/w of imatinib mesylate, 0.05-0.1% w/w of binder and 0% w/w of disintegrant, wherein the granulate composition is prepared using binder in isopropyl alcohol solvent.

In one of the embodiments according to present invention, it provides a granulate composition comprising imatinib mesylate crystalline Form-SA (I), which is stable, non-needle shaped and characterized by X-ray powder diffraction pattern comprising of at least five 2θ° peaks selected from 10.67, 12.90, 15.34, 19.49, 19.80, 26.06, 26.32 and 28.89±0.1 2θ°.

In one of the preferred embodiments according to present invention, relates to a pharmaceutical composition comprising crystalline non-needle shaped Form-SA of Imatinib mesylate and at least one or more pharmaceutically acceptable excipients. Such composition is substantially free of any other previously known crystalline forms of Imatinib mesylate.

In one of the embodiments according to present invention, relates to a pharmaceutical composition comprising crystalline non-needle shaped Form-SA of Imatinib mesylate, which is characterized by:
i. X-ray powder diffraction pattern comprising of at least five 2θ° peaks selected from 10.67, 12.90, 15.34, 19.49, 19.80, 26.06, 26.32 and 28.89±0.05 2θ°;
ii. X-ray powder diffraction pattern with absence of 2θ° peak at 25.08 2θ°;
iii. Non-needle shaped crystals;
iv. Particle size distribution of d₉₀<20 μm, d₅₀<10 μm and d₁₀<2 μm.
v. DSC data shows a melting point of between 222-224° C.

“d₁₀” value refers to particle size of 10% of active agent particles by volume is below the stated value.

“d₅₀” value refers to particle size of 50% of active agent particles by volume is below the stated value.

“d₉₀” value refers to particle size of 90% of active agent particles by volume is below the stated value.

In one of the embodiment according to present invention, it provides a process for making a granulate composition comprising imatinib mesylate, the process comprises wetting imatinib mesylate with a granulation liquid, which is preferably isopropanol containing binder, and granulating the mixture in a suitable granulator, e.g. high shear (Rapid mixer granulator/RMG) or fluid bed granulator (FBG or FBP), followed by drying, and optionally, sieving and/or milling, and further step involves addition of extragranular part of excipients and blending to make final blend for tablet compression, or filling into hard gelatin capsules.

In one of the embodiment according to present invention, it provides a slugging process for making a granulate composition comprising imatinib mesylate, the process comprises slugging of imatinib mesylate with at least one of the pharmaceutically acceptable excipients like disintegrants, binders, lubricants etc; followed by milling, sieving, and addition of extragranular excipients, and blending, to make final blend for tablet compression.

In one of the embodiment according to present invention, it provides a granulate composition comprising 90-99.95% w/w of imatinib mesylate, 0.05-0.2% w/w of polyvinyl pyrrolidone and 0-8% w/w of crospovidone, wherein the granulate is prepared by process comprising:
a) providing a solution of polyvinyl pyrrolidone in isopropyl alcohol;
b) wetting imatinib mesylate and crospovidone mixture with step a) solution, and
c) granulating the mixture in a granulator, followed by drying, and optionally sieving and/or milling.

In one of the preferred embodiments according to present invention, it provides a granulate composition comprising 95-99.95% w/w of imatinib mesylate, 0.05-0.2% w/w of polyvinyl pyrrolidone and 0-5% w/w of crospovidone, wherein the granulate is prepared by process comprising:
a) providing a solution of polyvinyl pyrrolidone in isopropyl alcohol;
b) wetting imatinib mesylate and crospovidone mixture with step a) solution, and
c) granulating the mixture in a granulator, followed by drying, and optionally sieving and/or milling.

In one of the preferred embodiments according to present invention, relates to a granulate and/or pharmaceutical compositions of imatinib mesylate, which are free of glidants.

In one of the embodiments according to present invention, relates to a process for making the pharmaceutical composition for oral administration comprising a step of admixing the imatinib mesylate granulate with pharmaceutically acceptable excipients, optionally, comprising a step of compressing the composition into tablets, and optionally followed by a film-coating.

In one of the embodiment according to present invention, it provides a process for making the pharmaceutical composition for oral administration comprising a step of admixing the imatinib mesylate granulate composition with pharmaceutically acceptable excipients, optionally, comprising a step of compressing the composition into tablets, followed by a film-coating, which may be by an aqueous or a non-aqueous film-coating.

In one of the embodiment according to present invention, it provides a process for making the pharmaceutical composition for oral administration comprising a step of admixing the imatinib mesylate granulate with pharmaceutically acceptable excipients, optionally, comprising a step of compressing the composition into tablets, followed by a non-aqueous film-coating, containing an organic solvent with an amount of less than about 60% v/v of water, preferably less than 50% v/v.

In one of the preferred embodiments according to present invention, it provides a process for making the pharmaceutical composition for oral administration comprising a step of admixing the imatinib mesylate granulate with pharmaceutically acceptable excipients, optionally, comprising a step of compressing the composition into tablets, followed by a non-aqueous film-coating, containing an organic solvent with an amount of less than about 10% v/v of water, preferably less than 5% v/v.

In one of the preferred embodiments according to present invention, the coating step may be carried using commercially available HPMC based coating materials for eg. Opadry brown 03F565018, the coating solution can be prepared with at least 90% v/v of C₁₋₄ organic solvents like isopropanol, ethanol etc, and <10% v/v of water. The coating of core tablets can be carried out in conventional pan coating apparatus.

In one of the preferred embodiments according to present invention, it provides a coating of core tablets using 95% IPA-5% water solution of Opadry brown 03F565018.

In one of the preferred embodiments according to present invention, it provides a process for preparing a pharmaceutical composition, preferably a tablet, containing imatinib, preferably imatinib mesylate wherein the pharmaceutical composition provides high polymorphic stability comprising: coating a pharmaceutical composition, preferably a tablet, comprising crystalline imatinib, with a coating solution, preferably a tablet coating solution, containing an organic solvent, for eg. C₁₋₄ alcohol solvent with an amount of less than about 10% v/v of water, preferably less than 5% v/v.

The term “pharmaceutical composition” or “oral pharmaceutical composition” or “oral dosage form” comprises capsule, tablet (film coated tablet, controlled release tablet, modified release tablet etc.), micro tablet, suspension, solution, dispersion, powder, granule and pellets. Capsules used as oral dosage form can be soft or hard capsules, though oral dosage form of the present invention is preferably tablet or capsule.

The term “about” is a value that can be considered +/−5% of the given value.

Based on context of discussion, the term “% w/w” refers to the relative value to total weight of granulate (or granules) or to total weight of pharmaceutical composition.

The term “Stable” in the context of the present invention refers to both physical stability and chemical stability. As used herein, the term “polymorphic stability” refers to the stability of imatinib to remain in the original polymorphic form without undergoing polymorphic conversion over time, for example, upon storage. As used herein, the term “storage” refers to a period of at least about 2 months. Preferably, storage is at 40° C./75% RH (relative humidity).

As used herein, the term “polymorphic conversion” refers to the conversion from a polymorphic form to any other polymorphic form of imatinib mesylate, such as conversion into any of forms H1, β, δ, [symbol] , I, II, V, X or amorphous form. In embodiments of the present invention the term “polymorphic conversion” refers to the conversion from a polymorphic Form-SA of imatinib mesylate to any of the above known forms. Preferably, form Polymorphic conversion is measured by techniques known in the art. In particular, each known polymorphic form of H1, β, δ, [symbol] , I, II, V, X or amorphous faint of imatinib mesylate amorphous form may be characterized by a unique set of PXRD or results from Differential scanning calorimeter.

In one of the embodiment according to present invention, it, provides a pharmaceutical composition comprising a crystalline non-needle shaped Form-SA of Imatinib mesylate, having HPLC purity of at least 99.8% and moisture content of less than 0.5%. The crystalline Form-SA of Imatinib mesylate can be obtained without any detectable impurities/contamination of any other previously known crystalline forms of Imatinib mesylate.

In one of the preferred embodiments of the present invention, it provides a solid oral pharmaceutical composition of imatinib mesylate comprising: Imatinib mesylate: 85-95% w/w, Binder: 0.05-0.2% w/w, Disintegrant: 6-15% w/w, Lubricant: 0.2-0.5% w/w, Film coating: <3% w/w; wherein the % w/w is relative to the total weight of pharmaceutical composition.

In one of the preferred embodiments of the present invention, it provides a solid oral pharmaceutical composition of imatinib mesylate comprising: Imatinib mesylate: 96-99% w/w, Binder: 0.05-0.2% w/w, Disintegrant: 0-5% w/w, Lubricant: 0.2-0.5% w/w, Film coating: 1-3% w/w; wherein the % w/w is relative to the total weight of pharmaceutical composition.

In one of the embodiment according to present invention, it provides granulate composition and/or the pharmaceutical composition of imatinib mesylate for its use in medicine, such as the treatment of various types of cancer diseases.

Binders include but are not restricted to polyvinylpyrrolidone, e.g. Povidone® K30 from BASF, starches, e.g. potato, wheat or corn starch; hydroxypropyl cellulose; hydroxyethyl cellulose and hydroxypropylmethyl cellulose, e.g. hydroxypropylmethyl cellulose-Type 2910 USP, hypromellose.

Suitable disintegrants according to the invention include but are not restricted to maize starch; CMC—Ca; CMC—Na; microcrystalline cellulose; cross-linked PVP, e.g. as known and commercially available under the trade names Crospovidone®, Polyplasdone®, available commercially from the ISP company, or Kollidon® XL; alginic acid; sodium alginate; and Guar gum. Preferably, Cross-linked PVP, e.g. Crospovidone® is used.

Film coating materials used in the composition include but are not limited to HPMC based coating materials e.g. Opadry brown 03B86854, Opadry Brown 03F565018 & Opadry Brown 02F86982 (Supplied by Colorcon Limited, USA).

As lubricants one or more of the following may be used Mg-, Al- or Ca-stearate, PEG 4000-8000 and/or talc. Preferably, magnesium stearate is used.

### EXAMPLES

### Example-1A

### Granulate Composition of Imatinib Mesylate

| | Quantity | | |
|---|---|---|---|
| | (mg/tablet) | | % w/w |
| | Granulate for | Granulate for | of granulate |
| INGREDIENTS | 100 mg/tab | 400 mg/tab | composition |
| Intragranular composition | | | |
| Imatinib Mesylate Form-SA | 119.50 | 478.00 | 92.04 |
| (equivalent to Imatinib) | | | |
| Crospovidone | 10.23 | 38.90 | 7.88 |
| Granulating fluid | | | |
| PVP K-30 | 0.10 | 0.39 | 0.08 |
| Isopropyl alcohol | q.s | q.s | — |
| Total | 129.83 | 517.29 | 100.00 |

### Manufacturing Process of Granulate Composition:

1. Imatinib Mesylate (Non-needle shaped) and Crospovidone are sifted through sieve #20.
2. PVP K-30 is dissolved in required quantity of Isopropyl alcohol.
3. Step-1 material is loaded into FBP and pre-mix step carried for 5 min and the granulating fluid of step-2 is sprayed onto the pre-mix material of FBP with proper fluidisation, and if required an additional amount of IPA is added.
4. The wet granules are dried at 30° C. until desired LOD got achieved.
5. The dried granules are passed through sieve #20.

### Example-1B

### Imatinib Mesylate Film Coated Tablet—FCT Prepared with Water Based Coating Composition

| | | | % w/w |
|---|---|---|---|
| | Quantity | | of |
| | (mg/tablet) | | coated |
| INGREDIENTS | 100 mg/tab | 400 mg/tab | tablet |
| Intragranular | | | |
| Imatinib Mesylate Form-SA | 119.50 | 478.00 | 87.62 |
| (equivalent to Imatinib) | | | |
| Crospovidone | 10.23 | 38.90 | 7.50 |
| Granulating fluid | | | |
| PVP K-30 | 0.10 | 0.39 | 0.07 |
| Isopropyl alcohol | q.s | q.s | — |
| Extragranular | | | |
| Crospovidone | 2.95 | 13.80 | 2.16 |
| Magnesium Stearate | 0.29 | 1.17 | 0.22 |
| Core tablet weight | 133.07 | 532.26 | 97.57 |
| Coating composition | | | |
| Opadry brown 03F565018 | 3.31 | 13.30 | 2.43 |
| Purified Water | q.s | q.s | — |
| Coated tablet weight | 136.38 | 545.56 | 100.00 |

### Manufacturing Process:

1. The dried granulate of Example-1A is taken for final blend preparation.
2. The weighed quantity of Crospovidone and Magnesium stearate are sifted separately through sieve #40 and sifted Crospovidone is added to the step-1 material and blended for 5 min, followed with lubrication stage blending with sifted Magnesium stearate for 5 min.
3. The final blend of step-2 is compressed with appropriate oval shape standard concave punches for 100 mg & 400 mg tablets respectively.
4. The Core tablets of step-4 are coated with aqueous dispersion of Opadry brown 03F565018. A processed placebo of Example 1B formulation containing Povidone, Crospovidone and magnesium stearate is prepared by the identical process as described above, and the processed placebo is used for XRD characterization in comparison to that of Example 1B.

### Example 2

### Granulate Composition of Imatinib Mesylate

| | Quantity (mg/tablet) | | | | |
|---|---|---|---|---|---|
| | Granulate for 400 mg/tab | | | | |
| | 1% | 2% | 3% | 4% | 5% |
| Ingredients | Crospovidone | Crospovidone | Crospovidone | Crospovidone | Crospovidone |
| Imatinib | 478.00 | 478.00 | 478.00 | 478.00 | 478.00 |
| Mesylate | | | | | |
| Form-SA | | | | | |
| (equivalent to | | | | | |
| Imatinib) | | | | | |
| Crospovidone | 5.00 | 10.00 | 15.00 | 20.00 | 25.00 |
| PVP K-30 | 0.39 | 0.39 | 0.39 | 0.39 | 0.39 |
| Isopropyl | q.s | q.s | q.s | q.s | q.s |
| alcohol | | | | | |
| Weight of | 483.39 | 488.39 | 493.39 | 498.39 | 503.39 |
| granulate | | | | | |
| composition | | | | | |

### Manufacturing Process of Granulate Composition:

1. Imatinib Mesylate (Non-needle shaped) and Crospovidone are sifted through sieve #20.
2. PVP K-30 is dissolved in required quantity of Isopropyl alcohol.
3. Step-1 material is loaded into FBP and pre-mix step carried for 5 min and the granulating fluid of step-2 is sprayed onto the pre-mix material of FBP with proper fluidisation, and if required an additional amount of IPA is added.
4. The wet granules are dried at 30° C. until desired LOD got achieved.
5. The dried granules are passed through sieve #20.

### Examples 3-7

### Imatinib Mesylate Film Coated Tablet—FCT Prepared with IPA-Water Based Coating Composition

| | Quantity | | | | |
|---|---|---|---|---|---|
| | (mg/tablet) | | | | |
| Ingredients | Example 3 | Example 4 | Example 5 | Example 6 | Example 7 |
| Intragranular | 1% | 2% | 3% | 4% | 5% |
| | Crospovidone | Crospovidone | Crospovidone | Crospovidone | Crospovidone |
| Imatinib | 478.00 | 478.00 | 478.00 | 478.00 | 478.00 |
| Mesylate | | | | | |
| Form-SA | | | | | |
| Crospovidone | 5.00 | 10.00 | 15.00 | 20.00 | 25.00 |
| PVP K-30 | 0.39 | 0.39 | 0.39 | 0.39 | 0.39 |
| Isopropyl | q.s | q.s | q.s | q.s | q.s |
| alcohol | | | | | |
| Granular | 483.39 | 488.39 | 493.39 | 498.39 | 503.39 |
| weight | | | | | |
| Extragranular | 5% | 4% | 3% | 2% | 1% |
| | Crospovidone | Crospovidone | Crospovidone | Crospovidone | Crospovidone |
| Crospovidone | 25.00 | 20.00 | 15.00 | 10.00 | 5.00 |
| Magnesium | 1.17 | 1.17 | 1.17 | 1.17 | 1.17 |
| Stearate | | | | | |
| Core tablet | 509.56 | 509.56 | 509.56 | 509.56 | 509.56 |
| weight | | | | | |

COATING COMPOSITION

| Opadry brown | 3% w/w of core tablet | | | | |
|---|---|---|---|---|---|
| 03F565018 | | | | | |
| Isopropyl | q.s | q.s | q.s | q.s | q.s |
| alcohol 95% - | | | | | |
| Water 5% | | | | | |
| Coated tablet | 524.85 | 524.85 | 524.85 | 524.85 | 524.85 |
| weight | | | | | |

| INGREDIENTS | % w/w of ingredients for Examples 3-7 |
|---|---|
| Imatinib Mesylate Form-SA | 91.07 |
| PVP K-30 | 0.07 |
| Crospovidone | 5.72 |
| Magnesium Stearate | 0.22 |
| Opadry brown 03F565018 | 2.96 |
| Coated tablet weight | 100.00 |

### Manufacturing Process:

1. The dried granulate of Example 2 is taken for final blend preparation.
2. The weighed quantity of extragranular portions of Crospovidone and Magnesium stearate are sifted separately through sieve #40, and sifted Crospovidone is added to the step-1 material and blended for 5 min, followed with lubrication stage blending with sifted Magnesium stearate for 5 min.
3. The final blend of step-2 is compressed with appropriate oval shape standard concave punches for 100 mg & 400 mg tablets respectively.
4. The Core tablets of are coated with coating solution (95% IPA-5% water) of Opadry brown 03F565018.

### Example 8

### Processed Placebo for Example 7 Formulation (Imatinib Mesylate FCT)

| | Quantity |
|---|---|
| Ingredients | (mg/tablet) |
| Crospovidone | 25.00 |
| PVP K-30 | 0.39 |
| Isopropyl alcohol | q.s |
| Intragranular weight | 25.39 |
| Crospovidone | 5.00 |
| Magnesium Stearate | 1.17 |
| Core tablet weight | 31.56 |
| Opadry brown 03F565018 | 3% w/w of core tablet |
| Isopropyl alcohol 95% - Water 5% | q.s |
| Coated tablet weight | 32.51 |

Manufacturing process: A processed placebo of Example 8 formulation containing Povidone, Crospovidone and magnesium stearate is prepared by the identical process as described above in Example 7, and the processed placebo is used for XRD characterization in comparison to that of Example 7.

### Example 9-13

### Imatinib Mesylate Film Coated Tablets (FCT)—SLUGGING and FCT Prepared with IPA-Water Based Coating Composition

| | Quantity | | | | |
|---|---|---|---|---|---|
| | (mg/tablet) | | | | |
| Ingredients | Example 9 | Example 10 | Example 11 | Example 12 | Example 13 |
| Intragranular | 1% | 2% | 3% | 4% | 5% |
| | Crospovidone | Crospovidone | Crospovidone | Crospovidone | Crospovidone |
| Imatinib | 478.00 | 478.00 | 478.00 | 478.00 | 478.00 |
| Mesylate | | | | | |
| Form-SA | | | | | |
| Crospovidone | 5.00 | 10.00 | 15.00 | 20.00 | 25.00 |
| PVP K-30 | 0.39 | 0.39 | 0.39 | 0.39 | 0.39 |
| Intragranular | 483.39 | 488.39 | 493.39 | 498.39 | 503.39 |
| weight | | | | | |
| Extragranular | 5% | 4% | 3% | 2% | 1% |
| | Crospovidone | Crospovidone | Crospovidone | Crospovidone | Crospovidone |
| Crospovidone | 25.00 | 20.00 | 15.00 | 10.00 | 5.00 |
| Magnesium | 1.17 | 1.17 | 1.17 | 1.17 | 1.17 |
| Stearate | | | | | |
| Core tablet | 509.56 | 509.56 | 509.56 | 509.56 | 509.56 |
| weight | | | | | |

COATING COMPOSITION

| Opadry brown | 3% w/w of core tablet | | | | |
|---|---|---|---|---|---|
| 03F565018 | | | | | |
| Isopropyl alcohol | q.s | q.s | q.s | q.s | q.s |
| 95% - Water 5% | | | | | |
| Coated tablet | 524.85 | 524.85 | 524.85 | 524.85 | 524.85 |
| weight | | | | | |

### Manufacturing Process:

1. Imatinib Mesylate, PVP K30 and Crospovidone are sifted together through sieve #20.
2. Magnesium Stearate is sifted separately through sieve #40.
3. Step 1&2 material is blended in blender for 10 min.
4. Step 3 material is subjected to slugging on a compression machine with 12.5 mm round punches.
5. The slugs obtained in step 4 are milled with 1.5 mm screen.
6. The granules of step 5 are sifted through sieve #20.
7. The granules and fines of step 6 material are separated by sifting the blend of step 6 through sieve #60.
8. Repeat the steps 5, 6 & 7 till granules to fines ratio obtained are 70:30.
9. The blend of step 8 is charged into blender.
10. The weighed quantity of Crospovidone is added to blend of step 9 and blended for 10 min.
11. Magnesium stearate is sifted through sieve #40 and added to the step 10 and blended for lubrication for 5 min.
12. The final blend of step 11 is compressed with punch size 15×8.00 mm oval shape standard concave punches.
13. The Core tablets of are coated with coating solution (95% IPA-5% water) of Opadry brown 03F565018.

### Example 13P

A processed placebo formulation containing Povidone, Crospovidone and magnesium stearate is prepared by the identical process as described above, and the processed placebo is used for XRD characterization in comparison to that of Example 13.

### Example 14

### Imatinib Mesylate-Film Coated Tablet—FCT Prepared with No Disintegrant and FCT Prepared with IPA-Water Coating

| | |
|---|---|
| | Quantity |
| Ingredients | (mg/tablet) |

Intragranular

| | | |
|---|---|---|
| Imatinib Mesylate | 119.500 | 478.000 |
| equivalent to Imatinib | | |
| Isopropyl alcohol | q.s. | q.s. |
| Povidone K-30 | 0.100 | 0.400 |

Extragranular

| | | |
|---|---|---|
| Magnesium Stearate | 0.400 | 1.600 |
| Tablet core weight | 120.00 | 480.00 |

| | |
|---|---|
| Opadry brown | 3% w/w of core tablet |
| 03F565018 | |
| Isopropyl alcohol | q.s |
| 95% v/v - Water 5% v/v | |

| | | |
|---|---|---|
| Coated Tablet weight | 123.60 | 494.40 |

| INGREDIENTS | % w/w of ingredients for Examples 3-7 |
|---|---|
| Imatinib Mesylate Form-SA | 96.68 |
| PVP K-30 | 0.08 |
| Crospovidone | 0.00 |
| Magnesium Stearate | 0.32 |
| Opadry brown 03F565018 | 2.92 |
| Coated tablet weight | 100.00 |

### Manufacturing Process:

The process of preparing Imatinib Mesylate granulate containing binder (Povidone K-30) with no disintegrant is prepared following the identical process as explained under IPA-Granulation process of Example 2.

The above obtained granulate is blended with extragranular lubricant (Magnesium stearate), followed by tablet compression with appropriate tooling. The tablet cores are coated to 3% w/w buildup with IPA-water (95:5) coating solution of Opadry brown 03F565018.

The physical properties of Example 14 granulate has adequate granule flow properties, good compressibility, no sticking/picking observed during compression. The coated tablet samples were also analyzed by DSC, and the result indicate a sharp endothermic peak of Form-SA at 223° C.

### XRPD Study Observations:

Example 1B Imatinib mesylate-Film Coated Tablets (IPA granulation and 100% aqueous coating), its corresponding API & placebo are subjected to XRD study. The XRD profiles comparison among Example 1B, and its corresponding API, indicate input API Form-SA retained in coated tablet and no contamination of Beta form of imatinib mesylate is observed. However, few additional peaks observed in Example 1B sample like 9.399, 9.838, 20.61 & 33.09.

Example 7 Imatinib mesylate-Film Coated Tablets (IPA granulation and 95% IPA-5% water coating), its corresponding API & placebo are subjected to XRD study. Example 7 Imatinib mesylate-Film Coated Tablets is subjected to directly exposed open petry dish (OPD), under stress stability conditions i.e at 40° C./75% RH, for two months. Initial and 40° C./75% RH 2M coated tablet samples XRD results of Example 7 indicate that Form-SA retained in the coated tablets, even after direct OPD exposure 2M samples, the samples retain input API Form-SA in coated tablet, and have no contaminations of any of reported forms H1, β, δ, [symbol] , I, II, V, X or amorphous form. Furthermore, there is no single additional peak observed in the initial sample XRD result of Example 7, when matched to its corresponding API XRD.

The above XRD interpretations and analogy indicate that the IPA-water Opadry coating is advantageous in comparison to that of 100% aqueous coating process, for the retention of Form-SA in coated tablet as well as free from polymorphic conversions or contaminations of reported crystalline forms of Imatinib mesylate.

### DSC Study Observations:

Example 7, 8, 13 & 13P coated tablet samples were subjected to OPD exposure alongside with Gleevec (Commercial sample), all initial and OPD exposure samples were analyzed by DSC. The DSC reports of Imatinib mesylate coated tablets of initial/OPD exposure samples of example 7, 8, 13 & 13P formulations have a sharp endothermic peak respectively, suggesting the drug melting point, approximately between 222-224° C. (Form-SA), in comparison to the Gleevec product having a sharp endothermic peak at 217° C. (β Form). This suggests the retention of Form-SA in coated tablet in the invention compositions of Imatinib mesylate.

While the foregoing pages provide a detailed description of the preferred embodiments of the invention, it is to be understood that the description and examples are illustrative only of the principles of the invention and not limiting. Furthermore, as many changes can be made to the invention without departing from the scope of the invention, it is intended that all material contained herein be interpreted as illustrative of the invention and not in a limiting sense.

1. A granulate composition of imatinib mesylate comprising 90-99.95% w/w of imatinib mesylate, 0.05-0.2% w/w of binder and 0-8% w/w of disintegrant.

2. A granulate composition according to the claim 1, wherein the imatinib mesylate is crystalline Form-SA (I), which is non-needle shaped and characterized by X-ray powder diffraction pattern comprising of at least five 2θ° peaks selected from 10.67, 12.90, 15.34, 19.49, 19.80, 26.06, 26.32 and 28.89±0.10 2θ°.

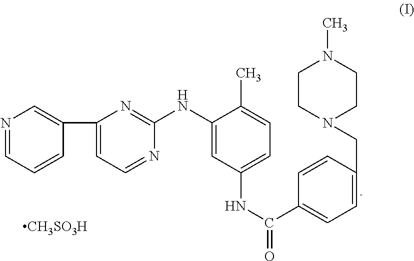

3. A granulate composition according to the claim 1, wherein binder is polyvinyl pyrrolidone and disintegrant is Crospovidone.

4. A granulate composition of imatinib mesylate comprising 95-99.95% w/w of imatinib mesylate, 0.05-0.2% w/w of binder and 0-5% w/w of disintegrant.

5. A granulate composition of imatinib mesylate according to claim-4, comprising 95-99.95% w/w of imatinib mesylate, 0.05-0.2% w/w of binder and 0% w/w of disintegrant, wherein the granulate composition is prepared using binder in isopropyl alcohol solvent.

6. A process of making a granulate composition according to claim 1, wherein the process comprising the steps of:
a) providing a solution of polyvinyl pyrrolidone in isopropyl alcohol;
b) wetting imatinib mesylate and crospovidone mixture with step a) solution, and
b) granulating the mixture in a granulator, followed by drying.

7. A process for making the pharmaceutical composition for oral administration of imatinib mesylate according to claim 6, comprising a step of admixing the imatinib mesylate granulate with pharmaceutically acceptable excipients.

8. The pharmaceutically acceptable excipients according to claim 7, are crospovidone and magnesium stearate and compressing into tablets.

9. A pharmaceutical composition for oral administration of imatinib mesylate comprising:
Imatinib mesylate: 96-99% w/w,
Binder: 0.05-0.2% w/w,
Disintegrant: 0-5% w/w,
Lubricant: 0.2-0.5% w/w,
Film coating: 1-3% w/w,
wherein the % w/w is relative to the total weight of pharmaceutical composition.

10. A pharmaceutical composition according to claim 9, wherein the coating of the tablet is carried out by using a C₁₋₄ alcohol solvent having less than 5% v/v water containing dissolved coating materials.
